(19) United States
(12) Patent Application Publication
(10) Pub. No.: US 2022/0096280 A1
(43) Pub. Date: Mar. 31, 2022

(54) REUSABLE NAPPY

(71) Applicant: Vladimir Sviridovich Medvedev, Dzerzhinsk (RU)

(72) Inventor: Vladimir Sviridovich Medvedev, Dzerzhinsk (RU)

(21) Appl. No.: 17/602,289

(22) PCT Filed: Apr. 8, 2020
(86) PCT No.: PCT/RU2020/000171

§ 371 Date: Oct. 8, 2021

(30) Foreign Application Priority Data
Apr. 9, 2019 (RU) 2019110566

(51) Int. Cl. A61F 13/49; A61F 13/15; A61F 13/62

(52) U.S. Cl. CPC A61F 13/49006; A61F 2013/1556; A61F 13/62; A61F 13/15268

(57) ABSTRACT

A reusable nappy configured in the form of side-openable pants with reusable fasteners is characterized in that the front part of the nappy is additionally provided with a waistband tightening arrangement disposed on the waistband of said front part in a space lying between fastening elements (see FIG. 6). The tightening arrangement can be configured in the form of two drawstrings disposed in channels on the waistband, which can be tightened and fastened in a knot, or in the form of other embodiments. The nappy can be provided with a front-detachable absorbent pad having a detachable barrier fabric, or with similar liners. Absorbent elements can be configured with a side-opening inward fold, resembling a side cap in appearance. A liner can be provided with a fold-over rear portion, inter alia, in the form of turndown collar. The aim of the invention is to provide an arrangement having fastenings which allows flexible adjustment of the size of the waistband and can be manipulated safely during fastening. The nappy is intended to have a training effect by virtue of slight wetness, but should not leak or allow excessive wetness. The arrangement is intended to allow easier and improved cleaning and to provide greater comfort for wearers and greater convenience for carers. The invention relates to reusable nappies and to clothing items for wearers susceptible to incontinence. Proposed are novel embodiments of a nappy and liners.

### FIELD OF INVENTION

The present invention relates to nappies, primarily to reusable nappies.

### BACKGROUND INFORMATION

Currently, the most widespread are disposable nappies, which are not burdensome to use for carers, but have a number of significant disadvantages. The number of economic disadvantages includes, first of all, a significant burden on the budget associated with one-time use, which results in a large number of consumed products. There are also hygienic factors that limit the use for the health of the carrier due to harmful chemicals that are part of the active absorbents, as well as due to low breathability, which causes an unfavorable over-dried and overheated microclimate in the inner space of the nappy that locks the lumbar-genital area of the user. There is also a real risk of infection of the genitourinary system of the carrier due to the practice of wearing a dangerously contaminated diaper after the first bowel movement in it. And it should be emphasized that such a danger is mixed with quite comfortable feelings of the user, what manufacturers are fighting for and what advertising campaigns are aimed at. A significant disadvantage of disposable nappies is a huge amount of highly polluted and infectious waste that is difficult to process, which has already become a serious social and environmental problem.

The use of reusable diapers is somewhat more troublesome for caregivers, but it allows using simple traditional solutions to eliminate the above-mentioned social, economic and environmental problems. In addition, the use of reusable nappies is gaining more and more supporters as a socially responsible behavior of people.

There is another side of the question. Until now, little attention has been paid to the problem of teaching the self-regulation of the nappy carrier (user), especially the newborn, who needs to form the skills of restraining natural needs from a clean slate. And for this, feedback is needed in the form of feelings of discomfort during spontaneous bodily functions of the body. That is, the nappy should get wet and naturally cool down due to the evaporation of moisture, which will create a training feeling of discomfort for the wearer.

At the same time, the nappy, if possible, should not get so wet and leak so as to pollute things in the neighborhood. However, almost all disposable diapers, as well as most sealed reusable diapers with a waterproof layer, exclude such discomfort, and therefore feedback, thereby depriving the user of up-to-date information and the appearance of a skill.

This leads to well-known common problems, for example, difficulties with teaching a child to use a pot, insufficient ability of an adult child to support natural functions, enuresis, etc.

One more psychological aspect can be noted—the formation of an incorrect primary skill, a behavioral standard in a newborn, namely the habit of sending natural needs at the first urge and the perception of clothes as a latrine. That is, parents are faced with the formation of incorrect skills, habits and ideas. The child learns and gets used to perceive clothes as a pot. And with the subsequent training in using the pot, the child simply does not understand what is wanted from him, since he has already been accustomed to comfortable defecation into clothes.

Before the era of disposable nappies, this set of modern problems was solved by itself—absorbent properties and permeability of the material of traditional nappies and swaddles provided a good microclimate and did not allow them to be used comfortably after contamination, and also gave reliable feedback.

Currently, the problem of feedback through discomfort can be solved by nappies without waterproof layers, for example, a nappy in the form of pants, described in the patent RU2400200[1]. These diapers are both educative, that is, giving feedback through feelings of discomfort after defecation, and giving the advantage of reusability, that is, they can be used repeatedly with the corresponding benefits, which we have already discussed. Pants made of multi-layer gauze also have the advantages of naturalness, there are no allergic reactions when using them, they absorb bowel movements well and quickly, they are easily rinsed, washed and dried, they can be steamed and ironed. This nappy is pulled together from the front on both sides with ribbons that are tied with a bow, which allows you to flexibly adjust the size of the belt and is aesthetically attractive. However, when using such a diaper, it is quite difficult to maintain a balance between getting wet and leaking, which leads to unpleasant cases of excessive getting wet, strong local leakage and contamination of other things and clothing. In addition, the fastening is not quite convenient, the ties must be dragged through the loops of the response surface when putting on, which is time-consuming.

To increase the capacity of the nappy in the form of pants, additional liners are used, which help to combat leakage, but this carries its own inconveniences—a larger nappy is required, an unfixed insert can shift, etc.

One of the most traditional variants of liners in diapers are traditional liners-diapers made of cotton gauze, folded in several layers and sewn in the form of squares, rectangles or triangles. Multi-layer gauze is a fairly comfortable and perfectly breathable material for liners in nappies-pants. Being a rarefied fabric, multilayer gauze forms honeycombs that absorb both liquid and suspended waste products very well. Traditional gauze square liners are usually folded twice or three times into an oblong rectangle and placed inside pants in the perineum.

This folding allows you to get a significant absorbency or capacity of the honeycomb. The use of additional liners in reusable diapers is convenient for users because it allows you to separate the most severe dirt and collect them on a simpler design and simply cleaned separate liner into the diaper. After use, a separate gauze liner is easy to rinse, wash, dry and iron, because it unfolds into a thinner and less layered plane.

However, such liners in nappies have disadvantages. Double or triple addition forms an excessive layer of material from the front and especially from the back edge with a noticeable thickening under the child's back, which causes unpleasant sensations and leads to skin rubbing(hurting). It should also be added that the intense jet when urinating into a loose material sometimes does not have time to disperse over the area and volume of the gauze liner, which leads to local leakage. You can also add that such a diaper with a liner is not optimal in design, because it does not allow you to easily and conveniently redistribute the material from less functionally loaded places to those places where a large absorbent capacity is required. In addition, the loose liner in the nappy can spontaneously shift from the optimal position, which is especially important for actively moving children.

For the manufacture of reusable nappies and liners in modern practice, not only cotton gauze is used. Recently, there has been a significant increase in attention to reusable hygroscopic hygiene products and materials for their manufacture. For example, the Chinese patent WO2017114974 presents a textile recoverable material for the manufacture of hygiene devices [2] for incontinence, to the fibers of which one or more antimicrobial and/or hydrophilic and/or stain-releasing agents are attached.

The closest to the claimed solution is a reusable diaper described in US patent US2014221954[3], which is an external nappy consisting of a back part with snap buttons for fastening the nappy waistband on the side wings of the waist part of the nappies, a perineum and an expanded front part with corresponding parts of the snap buttons. The outer nappy has a barrier layer. Inside, the nappy is equipped with internal pockets and valves with liners that can be placed and fixed in various versions. The size of the nappy waistband is adjusted step-by-step with the help of a numerous point response fasteners in the area of the carrier's abdomen.

In one of the variants of this solution, the diaper is equipped with a valve for fixing the liner. At the same time, the valve has the shape of a pocket and has an extension forward, fixed at the corners of the front edge to the outer nappy with push-button fastening method. Under this valve (between it and the external nappy), the main sorbing liner is placed, which is located at the back in the pocket, in the middle under the valve, and is fixed in front through holes at the same points of reusable attachment as the valve itself to the external nappy. This solution has the following disadvantages.

First, let's consider the disadvantages of attaching a diaper of this design to the waistband. The construction is oversaturated with fastening buttons on the stomach, which makes it unnecessarily complex and time-consuming. In addition, when manipulating the fixing or detaching of point fasteners on the abdomen, it is necessary to press on the stomach, which will at least cause unpleasant sensations to the user, and in some cases is generally unacceptable. At the same time, the adjustment of the waistband size is step-by-step, that is, inaccurate, which leads to the mandatory use of elastic elements. Step-by-step rigid adjustment also leads to the fact that it is difficult to put a nappy on if it is collected in pants before putting them on the user.

Second, we will analyze the disadvantages of the considered nappy related to the design of the barrier layer and absorbent liners. A moisture-proof layer of the diaper is placed in the outer surface of the pants, which will block the space on the carrier, and deprive it of feedback, that is, the diaper can not be attributed to educative (training) Such a diaper can not be disinfected by washing with boiling and ironing with an iron.

In addition, in this solution, we also see the presence of inseparable absorbent layers and barrier layers, which, when cleaning the product, will cause the appearance of dead-end zones that are difficult to disinfect. Since the valve to the main nappy is sewn in the form of a pocket, it is difficult to wash and dry the nappy, which also complicates disinfection by ironing with a hot iron. The valve does not have a loose cellular structure and is a barrier on the way to the liner and prevents the absorption of excretions into the main liner located behind the valve. The liner is placed under the valve, which requires its special fastening. The requirement of special fasteners sharply narrows the choice of liners to special liners with holes. The valve itself does not have a sufficient absorption capacity.

The purpose of this invention is to offer a reusable nappy device with a learning effect by providing feedback during the event of incontinence. At the same time, the design of the diaper should ensure safe and gentle manipulation when putting it on the user, it should be convenient for both the user and the guardians. When putting on a nappy, the size of the nappy's waistband should provide the possibility of smooth adjustment. The length of the perineum of the nappy should be regulated by folding, and the configuration obtained during addition should be comfortable and utilitarian inside, aesthetically and structurally attractive outside. The diaper should prevent leakage or heavy soaking and contamination of the surrounding clothing, underwear and linen. The nappy should provide a reliable and convenient attachment of additional absorbent materials elements, allow the expansion of its functionality due to the possibility of using a wide selection of liners and similar sorbing devices and materials from different manufacturers, including disposable, reusable, with special properties, etc. The liner itself in the nappy should be simply, conveniently and securely attached both in front and behind. When fixing the liner in the back of the diaper, the fastening elements should not cause unpleasant sensations when the wearer is lying on his back. The nappy should be cleaned quite simply and conveniently and completely restore its hygienic properties by rinsing, washing, ironing and drying.

### DESCRIPTION OF THE INVENTION

The technical solution proposed in the invention proceeds from a new view and a new approach of the author to the problems of using nappies, the formation of healthy reactions and habits in newborns, and in general to the problem of incontinence.

Further aspects and features of the technology of this application will become obvious from the detailed description provided below. In addition, any one or more aspects of the technology of this application may be implemented individually or in combination with any one or more other aspects of the technology of this application. It should be understood that the detailed description and examples given in it are intended for illustration and should not be considered as limiting.

To achieve these goals, a diaper is offered, made in the form pants detachable on the sides (see FIG. 1). The nappy has an expanded rear part (1) with side extensions of the waistband (2), each of which is equipped with at least one reusable fastening element (3) of the waistband, the perineum (4) and an expanded front part (5). The front part (5) of the nappy is equipped with reciprocal reusable fastening elements (6) of the waistband located on the sides of the expanded front part. At the same time, the diaper is additionally equipped with a tightening structure (7) located on the front of the nappy, and designed for smooth and convenient adjustment of the size of the waistband on the carrier.

In one or more variants (see FIG. 2), the tightening structure (7) is located on the waistband of the diaper of the front part of the diaper in an increased space A, between the reciprocal reusable fastening elements (8) of the fastenings of the waistband of the nappy (the waist part of the pants) of the nappy. The reciprocal fastening elements are located on the front part symmetrically on both sides of the abdomen. As elements of fastening of the waistband of the nappy (the waist part of the pants), point fastening elements can be used, for example, snap fasteners in the form of snap buttons (8). The space between the snap buttons on the front part can be more than 10 cm, which will ensure that the waistband of the nappy is spanned by 3 cm or more, with a slight plication of the fabric. This is necessary (see FIG. 3) in order to provide a space (9) between the assembled waistband and the body for safe manipulations with the fingers of the hand (10 and 11) when fastening the waistband of the diaper. That is, the waistband if the nappy is assembled on the waist of the user must have a space sufficient for placing the finger of the hand (10) in order to make a snapping of the fastener without touching or pressing the hand on the body or abdomen of the user.

In one or more versions of the diaper (see FIG. 2) the tightening structure can be made symmetrically to the midline of the abdomen, for example, in the form of two pieces (12) of a braid or cord passing through the channels (14) inside the waistband. The far ends (13) of pieces are attached to the nappy. The places where the ends of the braid are fixed to the outer fabric of the nappy are located closer to the snap buttons (8), the braid pieces themselves are passed through the channels (14) under the fabric towards the midline of the abdomen and go out through the holes (15) in the fabric. Exit holes (15), for example, made in the form of loops, are located symmetrically on both sides of the median line of the abdomen, for example, at a distance of 2-4 cm. In other versions, both ends of cords can exit, for example, through a single hole located in the center, etc. From the outside, the cords can be attached separately, for example, with the help of clamps, providing the required pulling of the cord from the hole (15) and thereby the required assembly of the tissue of the channels (14) and, accordingly, the front part of the nappy. In another case (see FIG. 4) the ends of the cord can be elongated and attached to each other, for example, into the node (16). As a result, the desired size is provided when fitting the waistband of the nappy on the user. The waistband of the nappy can also be additionally equipped with various elastic elements (17).

The tightening structure can also be made in other versions known to industry specialists, for example (see FIG. 5) in the form of straps with buckles (18), lacing, etc.

In one or more variants (see FIG. 6), the nappy on each side of the wing of the back part of the nappy waistband (2) has two vertically arranged snap fasteners (19), for attaching the waistband of the nappy on both sides to the corresponding vertical pairs of snap fasteners (20) located on the sides of the front part (5) of the waistband of the nappy. For step-by-step adjustment of that size of the waistband of the nappy, one or more alternative pairs of response buttons (21) can be additionally placed on each side of the front part, located symmetrically and one step closer to the midline of the abdomen, but so that an elongated space remains for placing the tightening structure (7).

In one or more variants (see FIG. 6), the diaper can be additionally equipped with absorbent internal elements (46) in various versions—in the form of liners, in the form of absorbent pads, etc. For example, the absorbent element can be made in the form of a pad (46), which is sewn to the main nappy from the inside along the line (22), and then attached to the nappy with snap buttons (23).

In one or more variants, the nappy may have point elements (24) on the inner surface of the front for attaching a liner or an absorbent pad. The liner at the back may not have special elements for mounting and can be freely placed behind under the back of the user. This option may be convenient for non-moving recumbent users, for example, for newborns.

The absorbent element in the form of a gasket (46) or a liner (see FIGS. 7 and 8) may have a special barrier cloth element (25) attached distantly of the carrier, designed to prevent the leakage through the absorbent element of the gasket (46) or the liner. The barrier cloth element (25), for example, can be permanently attached to the absorbent pad or to the liner at the back, and at the front to reusable fasteners, for example, snap buttons (26). Accordingly, the response buttons (27) are placed on the liner or on the absorbent pad. Thus, in the unfastened state, the barrier canvas element in the front will be unfastened from the liner, but attached to the liner or to the nappy with an absorbent pad at the back, and only in one line, which allows you to effectively and conveniently rinse, wash, dry and iron the liner itself or the absorbent pad and the nappy. The barrier canvas element can be made of various materials, for example, from a waterproof fabric with a polyurethane layer, etc. In shape, it can repeat the shape of the liner itself, or it can be slightly smaller in order to prevent its contact with the skin of the carrier. For the purpose of ventilation, the barrier cloth element can have local perforation (28) in the areas of the lowest barrier load to improve ventilation, for example, in corners, along the perimeter, etc. The barrier cloth element can also have elastic zones, both for tightening it and holding the liner, and for its surface configuration to obtain the desired bulges, barriers, gaps, etc.

In one or more variants, the special barrier cloth element may have special holes (29) in the front, located respectively to the snap-fasteners (27) in front of the liner itself or the absorbent pad to the nappy. In this case, the attachment of the liner to the nappy is carried out through these holes, through which the snap-fasteners for attaching the liner (27) or gaskets to the outer nappy are passed.

The liner (see FIG. 8) or the absorbent gasket may have a design with a longitudinal inner pocket (30) between the absorbent walls (31) on one of the sides, similar to a pilot hat. This will facilitate cleaning operations and allow, if necessary, to easily and reliably place additional absorbent elements inside—disposable, reusable, with special properties (hygienic, therapeutic, sorption, aromatic, etc.).

In one or more versions, the nappy liner can be made of various shapes, for example, rectangular, oval, trapezoidal, in the form of an hourglass and can be configured in accordance with the internal space of the nappy of a specific drawing.

In one or more versions (see FIG. 9) the nappy liner can additionally have a rear mount in the form of a flip-back (32) in various versions. The backdrop can be solid, can be composite, can be attached to the liner, for example along the lines (33), can be permanently attached or with the possibility of detaching. For example, the backdrop can be made in the form of two straps (34) or in the form of a non-thick fabric extension with straps (35), which are provided at the ends with reusable fasteners (36) for fixing on the exterior of the nappy. Straps (see FIG. 10) can pass from the outside along the waistband of the nappy forward through the belt loops (37) on the waistband and then be attached to reusable fasteners (36), for example, snap-fasteners or snap buttons. In the latter case, the nappy is additionally provided with belt loops (37) in the waistband from the outside. In the simplest version (see FIG. 9), the flip-back can be made in the form of two ribands (38) that go out above the waistband of nappy and are tied to it, for example, by its loops or belt loops.

The backing folds (see FIG. 9) over the top of the waistband of the diaper along the line P1, which can run either above the rear edge (39) of the liner, or below. In the latter case, the liner will itself be pressed against the waistband of the diaper and will protrude outward above the waistband of the nappy. The flip-over back supports and fixes the rear end of the insert by pinching the backdrop by the nappy waistband pressed against the body and by adhering to the nappy waistband, which is pinched in the backdrop folded through it. The back can be additional reusable fasteners (36), for example, snap buttons, loops, etc., for fixing outside the nappy. In this case, the outside side of the diaper is provided with reciprocal reusable fasteners, such as snap-fasteners, snap buttons, etc.

In one or more versions (see FIGS. 9 and 11), the flip back of the liner can be made according to the type of turn-down collar (40) of various configurations. Such a design will be intuitive to the user and convenient, for example, when putting on a nappy, when the liner is pre-fixed on the snap buttons in front, and the back is held on both sides by the hands of the waistband of the nappy with the turn-down collar turned over through it. This option allows you to visually control the rear position of the liner and easily correct it if necessary. The turn-down collar can be equipped with fastening elements from the outside to the nappy, for example, snap-fasteners or loops (41) under the buttons (42) on the waistband of the nappy.

The flip back can carry an independent aesthetic or informational function. It can be furnished with drawings or other decorations. Information about the state of the interior medium of a nappy, etc.

In one or more variants (see FIG. 12) the nappy may have elements on the outside for attaching folds in front, formed if necessary to reduce the size of the perenium.

These can be pairs of snap buttons (43 and 44) located symmetrically on the sides of the middle line (45). At the same time, these elements can be arranged in the form of a trapezoid with a smaller side (KM) on top. When adding such a figure (see FIGS. 13, 14), the folds on the nappy are formed in the form of a smile. If such a fold is straightened forward (see FIG. 15), then it turns out a bulge that attracts attention. This can be used for advertising purposes and for promotion purposes. In addition, the resulting bulge from the inside is a niche that can be convenient for placing the genitals in the case of male nappy, as well as for placing additional functional elements in the niche, for example, absorbent, therapeutic, for sampling for analysis, etc.

### BRIEF DESCRIPTION OF THE DRAWINGS

The drawings described here are intended for illustrative purposes and are not intended to limit the volume or spirit of the technology of the present invention.

FIG. 1. A nappy with fasteners in an open form with a variant of a tightening design.

FIG. 2. A nappy on snap fasteners with a tightening design in the form of pieces of the ribbon passed through the channels on the belt

FIG. 3. An explanation of the manipulation with the bending of the nappy waistband with span button when fastening the closing button.

FIG. 4. A front view of the nappy assembly with the waistband tightened with the help of cord pieces. Outside, the ends of the cords are tied into a knot with a bow.

FIG. 5. Front view of the nappy assembly with a waistband of the nappy tightened with a strap, fixed with a buckle.

FIG. 6. A variant of the nappy in the open form with pairs of snap buttons on the sides, with an absorbent pad sewn on the back, and attached to the nappy on the snap buttons in front.

FIG. 7. A variant of the nappy with an absorbent pad, additionally equipped with a waterproof cloth on the side of the nappy.

FIG. 8. A variant of the liner into the nappy, with mounting only on the buttons in front. The liner has an internal longitudinal cavity according to the type of pilot hat and is additionally equipped with a barrier cloth permanently attached to the back, and in front it has special holes for attaching to the snap buttons of the liner and the nappy, as well as perforation.

FIG. 9. Some variants of the liner with a flip back.

FIG. 10. Views of the front and back of the nappy assembly with a flip back, the straps of which are passed through the loops specially made on the waist part of the nappy and attached to the nappy with snap buttons on the outside.

FIG. 11. Rear view of the nappy with the liner assembly. The liner is equipped with a flip back in the form of a turn-down collar, the tips of which are attached to the nappy waistband with buttons.

FIG. 12. The scheme of the nappy assembly and the scheme of assembling the buttons to obtain the desired relief. The front view of the nappy is shown with buttons for changing the size by means of fixed folds that tighten the perineum in front. The snap buttons are arranged in the form of a trapezoid.

FIG. 13. Photo of the front of the nappy assembly on a user with snap buttons for changing the size of the perineum in the open form, in the form of a trapezoid. View before the assembling with folds formation.

FIG. 14. Photo from the front of the nappy assembly on a carrier with snap buttons for changing the size of the nappy in perineum, assembled, the snap buttons are placed on the nappy in the form of a trapezoid, with a minimum size. The folds are gathered in the form of a smile.

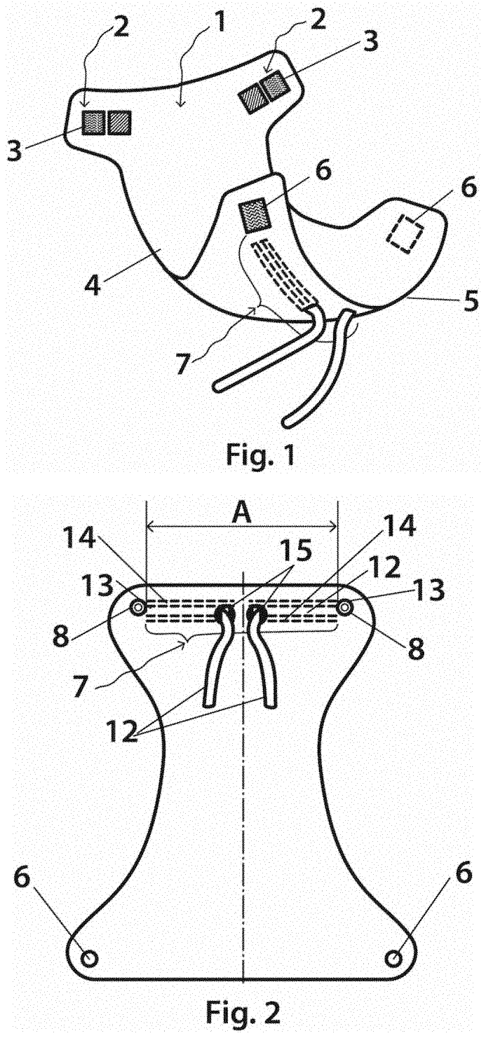

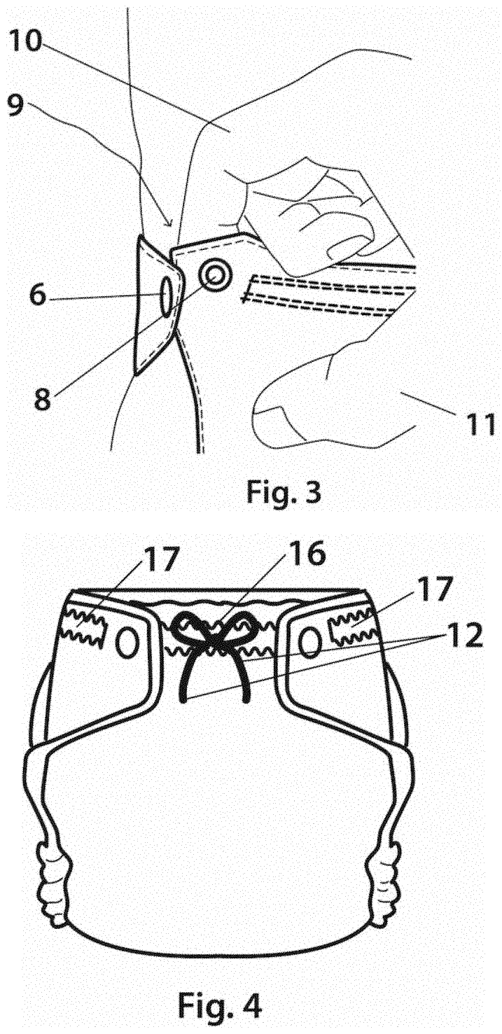

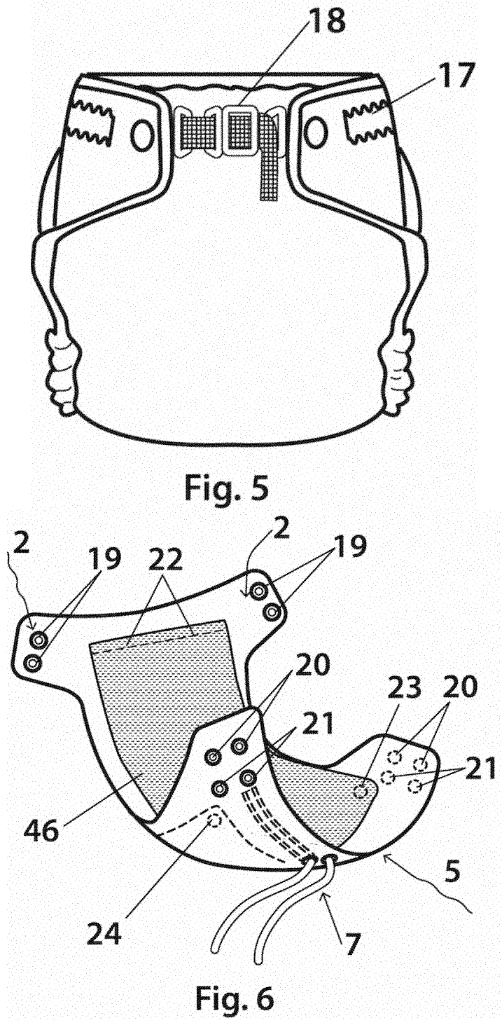

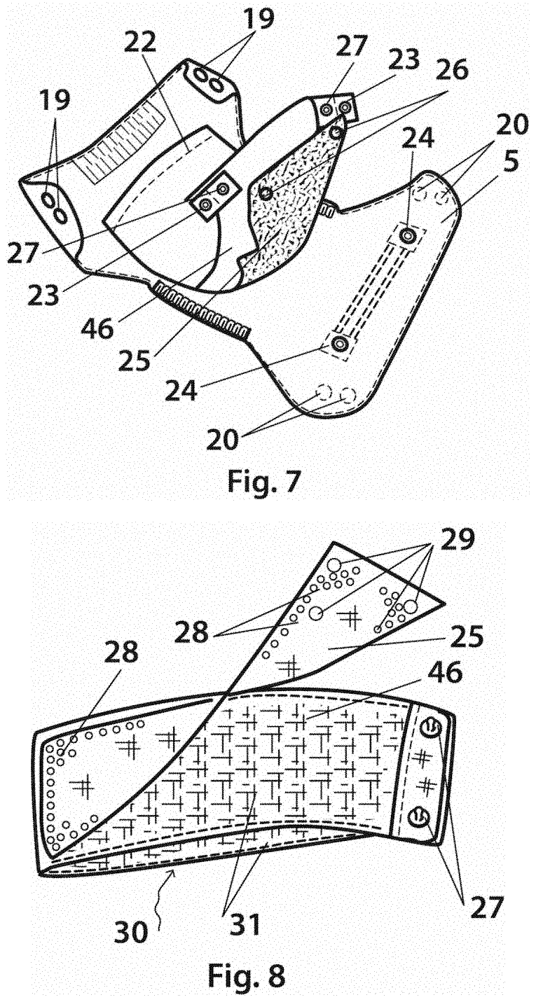

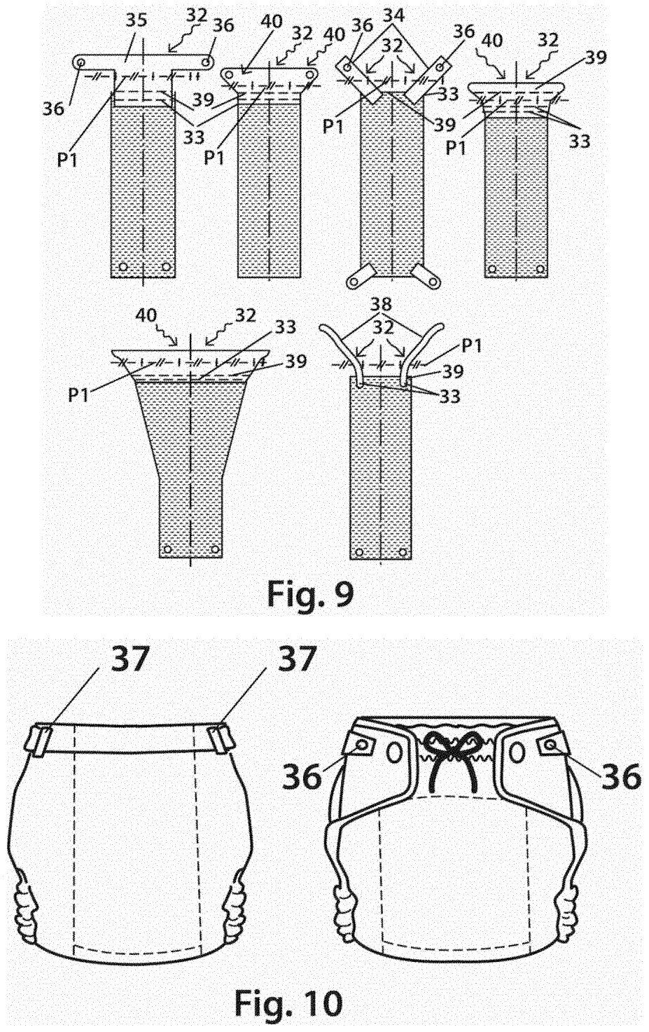

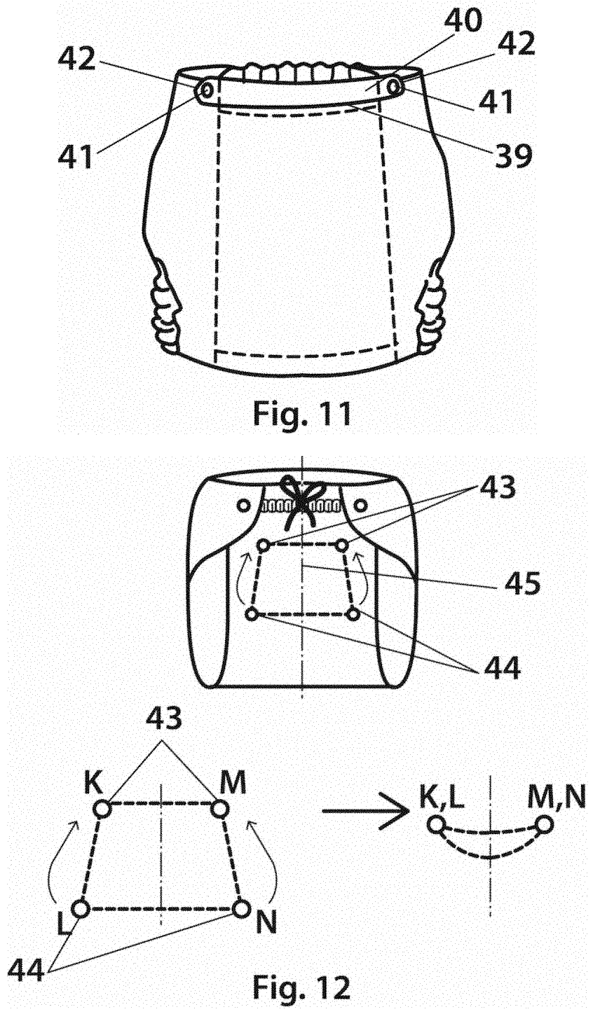

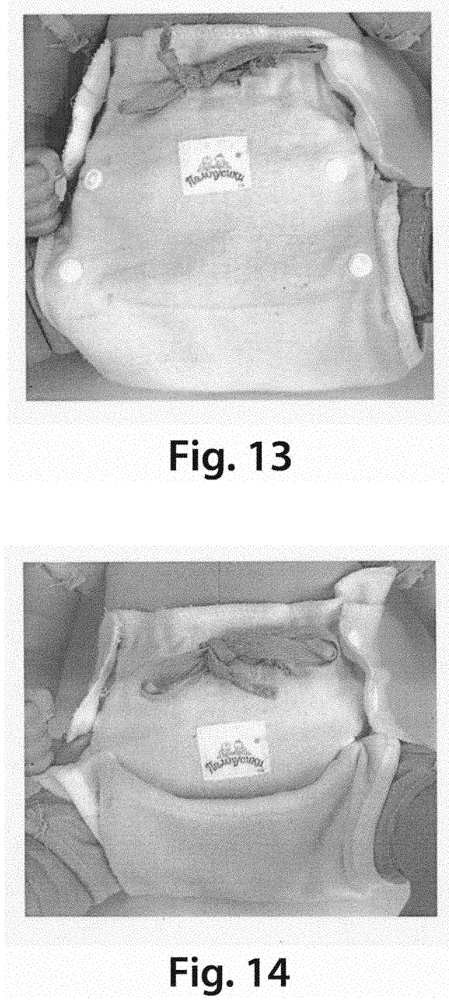

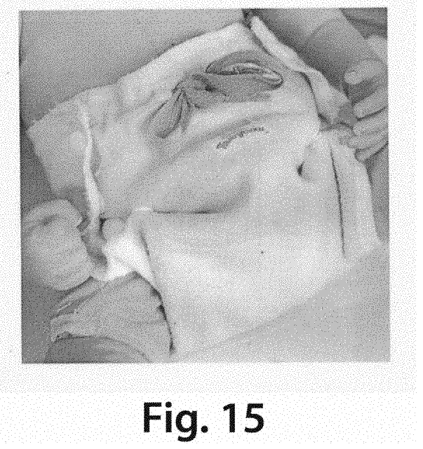

FIG. 15. Photo of the front of the nappy assembly on a carrier with buttons for changing the size of the perineum, located in the form of the trapezoid in assembled form, that is, with a minimum size. The folds are straightened forward to form an internal cavity.
[1] Patent of the Russian Federation RU2400200;
[2] Patent WO2017114974;
[3] USA Patent US2014221954.

1. A reusable nappy made in the form of pants detachable on the sides, consisting of an expanded back part with side wings of the waistband of the nappy, and each of the side wings is equipped with at least one reusable attachment element for attaching the back of the nappy to the front of it, the perineum and an expanded front part equipped with matching attachment elements of the waistband of the nappy, characterized in that the front part is additionally equipped with a tightening structure, which is located on the waistband of the front part in the zone between the matching attachment elements of the waistband of the nappy.

2. A reusable nappy of claim 1, characterized in that the tightening structure is located in an increased zone between the reciprocal elements of the attachment of waistband of the nappy, which are located symmetrically on both sides of the abdomen.

3. A reusable nappy of claim 2, characterized in that snap buttons are used as elements of a reusable attachment of the waistband of the nappy, the size of the close-ended waistband of the nappy is larger than the size of the waist of the carrier by at least 3 cm, and the increased distance between the snap buttons and, accordingly, the tightening structure in the stretched state have a length of at least 10 cm.

4. A reusable nappy of claim 3, characterized in that the tightening structure is made in the form of two pieces of braid passing under the fabric in the channels, with one of the ends of each segment fixed closer to the snap button, and the other end of each segment goes to the outside of the nappy through a hole located in the midline of the abdomen.

5. A reusable nappy of claim 4, characterized in that the ends of the pieces of the braid that go out to the outside of the nappy through the hole are elongated, with the possibility of being pulled together and fastened to each other on a knot.

6. A reusable nappy of claim 4, characterized in that the elements of reusable fastening of the waistband located on each side extension of the rear part are made in the form of two vertically arranged snap buttons, and the corresponding elements of the front part are made in the form of at least two pairs of snap buttons.

7. A reusable nappy according of claim 1, characterized in that the front part of the nappy is provided with snap buttons from the inside for attaching the liner.

8. A reusable nappy of claim 7, characterized in that the nappy is additionally equipped with elements, which located outside the nappy for attaching the flip back of the liner.

9. A reusable nappy of claim 8, characterized in that the elements for attaching the flip back of the liner are made in the form of snap buttons and are placed on both sides on the sides of the waistband of the back of the nappy.

10. A reusable nappy of claim 7, characterized in that it is additionally equipped with an absorbent pad permanently attached from the inside to the back of the nappy and equipped with appropriate snap buttons in front for attaching from the inside to the front of the nappy.

11. Reusable nappy of claim 10, characterized in that the absorbent pad is additionally equipped with a waterproof cloth located on the side, at the distance from the body of the user, and in the rear part the waterproof cloth is permanently attached and in a straight line, and in front it is attached to snap buttons.

12. A reusable nappy of claim 10, characterized in that the absorbent pad is additionally equipped with an internal cavity, the entrance to which is made from one of the side edges.

13. A reusable nappy of claim 1, characterized in that it is made with the possibility of changing the length of the perineum by forming a horizontal fold in front, for which it is additionally equipped with at least two pairs of snap buttons located symmetrically to the middle line of the abdomen, but at different distances from the waistband.

14. A reusable nappy of claim 13, characterized in that the upper symmetrical two snap buttons are located at a smaller distance from each other than the lower ones.
